(19) United States
(12) Patent Application Publication
(10) Pub. No.: US 2006/0110358 A1
(43) Pub. Date: May 25, 2006

(54) Combination therapy for treatment of fibrotic disorders

(76) Inventor: Henry H. Hsu, Hillsborough, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/526,087

(22) PCT Filed: Aug. 20, 2003
(86) PCT No.: PCT/US03/26135

§ 371 Date: Sep. 14, 2005

Related U.S. Application Data
(60) Provisional application No. 60/406,901, filed on Aug. 28, 2002.

(51) Int. Cl. A61K 38/21

(52) U.S. Cl. 424/85.6

(57) ABSTRACT

The present invention provides methods of treating a fibrotic disorder. The methods generally involve administering an effective amount of IFN-γ and an effective amount of pirfenidone or a pirfenidone analog.

### FIELD OF THE INVENTION

This invention is in the field of therapy of treating fibrotic diseases.

### BACKGROUND OF THE INVENTION

Current data indicate that fibrosis is not a static process; extracellular matrix is constantly being laid down and resorbed and the progressive accumulation of fibrous tissue is thought to represent a relative imbalance between pro-fibrotic processes and anti-fibrotic processes. If these processes are not properly regulated, the pathologic and progressive accumulation of collagen in the extracellular space as a result of a disordered wound healing process leads to replacement of normal cells by dense fibrous bands of protein, and results in fibrotic disease with disordered function in the affected organ (for example, impairment of respiratory function, impaired circulatory function via fibrotic changes in arterial walls, fibrotic degeneration of renal and liver function, degenerative musculoskeletal function, fibrotic degeneration of cardiac muscle or skeletal muscle, fibrotic degenerative changes in neuronal tissues in the central nervous system as well as the peripheral nervous system, etc.).

Pulmonary fibrosis can be caused by a number of different conditions, including sarcoidosis, hypersensitivity pneumonitis, collagen vascular disease, and inhalant exposure. The diagnosis of these conditions can usually be made by careful history, physical examination, chest radiography, including a high resolution computer tomographic scan (HRCT), and transbronchial biopsies. However, in a significant number of patients, no underlying cause for the pulmonary fibrosis can be found. These conditions of unknown etiology have been termed idiopathic interstitial pneumonias or idiopathic pulmonary fibrosis. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

The logic of dividing idiopathic interstitial pneumonias into these categories is based not only on histology, but also on the different response to therapy and prognosis for these different entities. DIP is associated with smoking and the prognosis is good, with more than 70% of these patients responding to treatment with corticosteroids. NSIP patients are also frequently responsive to steroids and prognosis is good, with 50% of patients surviving to 15 years. In contrast, the UIP histologic pattern is associated with a poor response to therapy and a poor prognosis, with survival of only 3-5 years.

Idiopathic pulmonary fibrosis (IPF) is the most common form of idiopathic interstitial pneumonia and is characterized by the UIP pattern on histology. IPF has an insidious onset, but once symptoms appear, there is a relentless deterioration of pulmonary function and death within 3-5 years after diagnosis. The mean age of onset is 60-65 and males are affected approximately twice as often as females. Prevalence estimates are 13.2-20.2 per 100,000. The annual incidence is estimated to be 7.4-10.7 per 100,000 new cases per year.

Published evidence suggests that less than 20% of patients with IPF respond to steroids. In patients who have failed treatment with steroids, cytotoxic drugs such as azathioprine or cyclophosphamide are sometimes added to the steroid treatment. However, a large number of studies have shown little or no benefit of these drugs. There are currently no drugs approved for treatment of IPF.

Fibrosis of the liver occurs due to a chronic toxic insult to the liver such as hepatitis C virus (HCV) or hepatitis B virus (HBV) infection, autoimmune injury, and chronic exposure to toxins such as alcohol. Chronic toxic insult leads to repeated cycles of hepatocyte injury and repair accompanied by chronic inflammation. Over a variable period of time, abnormal extracellular matrix progressively accumulates as a consequence of the host's wound repair response. Left unchecked, this leads to increasing deposition of fibrous material until liver architecture becomes distorted and the liver's regenerative ability is compromised. The progressive accumulation of scar tissue within the liver finally results in the histopathologic picture of cirrhosis, defined as the formation of fibrous septae throughout the liver with the formation of micronodules.

Renal fibrosis is a complication of kidney injury and can contribute to organ failure. Tubulointerstitial and glomerular fibrosis is a morphologic hallmark of chronic, progressive renal disease and is thought to be the final common mechanism leading to end-stage renal disease. There are multiple etiologies of renal fibrosis. In particular, Type I and II diabetes mellitus are common causes of renal fibrosis. In addition, there are toxic, drug-induced, metabolic, structural, genetic, and infectious causes of chronic renal insufficiency related to renal fibrosis. In a number of pathologic conditions, the etiology is unknown. Of particular clinical relevance, the rate of decline of the glomerular filtration rate in patients with chronic renal disease correlates strongly with the extent of tubulointerstitial and glomerular injury. Tubulointerstitial fibrosis is also a component of age-related structural changes in otherwise normal kidneys and is a hallmark of chronic allograft nephropathy (chronic allograft rejection), the most common cause of kidney transplant failure in the first decade after transplantation. Accumulation of proteins, such as fibronectin and various collagens, in the interstitium of the kidney is thought to be a fundamental process in development of tubulointerstitial scarring. Increased synthesis, decreased degradation, or both can underlie interstitial protein accumulation. During fibrosis, interstitial fibroblasts proliferate and are primarily responsible for increased production of interstitial proteins. Currently, there are no drugs that adequately treat renal fibrosis.

In addition to fibrotic disorders of the lung, liver and kidney, many other organs and tissues are susceptible to fibrotic degeneration. In particular, cardiac injury from hypoxia or ischemia, toxins, infectious agents, genetic etiologies, and structural disorders can lead to an inappropriate chronic wound healing process that results in fibrosis of cardiac tissue.

There is a need in the art for methods of treating fibrotic disorders. The present invention addresses this need.

### LITERATURE

WO 01/34180; Ziesche et al. (1999) N. Engl. J. Med. 341:1264-1269; du Bois (1999) N. Engl. J. Med. 341:1302-1304; U.S. Pat. No. 6,294,350; EP 795,332; King (2000) N. Engl. J. Med. 342:974-975; Ziesche and Block (2000) Wien. Klin Wochenschr. 112:785-790; Raghu et al. (1999) Am. J. Respir. Crit. Care Med. 159:1061-1069; Stem et al. (2001) Chest 120:213-219; Gay et al. (1998) Am. J. Respir. Crit. Care Med. 157:1063-1072; Dayton et al. (1993) Chest 103:69-73.

Al-Bayati et al. (2002) Biochem. Pharmacol. 64:517-525; Shihab et al. (2002) Am. J. Transplant. 2:111-119; Yu et al. (2002) Curr. Opinion Pharmacol. 2:177-181; U.S. Pat. Nos. 5,310,562; 5,518,729; 5,716,632; and 6,090,822.

METAVIR (1994) Hepatology 20:15-20; Brunt (2000) Hepatol. 31:241-246; Alpini (1997) J. Hepatol. 27:371-380; Baroni et al. (1996) Hepatol. 23 :1189-1199; Czaja et al. (1989) Hepatol. 10:795-800; Grossman et al. (1998) J. Gastroenterol. Hepatol. 13:1058-1060; Rockey and Chung (1994) J. Invest. Med. 42:660-670; Sakaida et al. (1998) J. Hepatol. 28 :471479 ; Shi et al. (1997) Proc. Natl. Acad. Sci. USA 94:10663-10668; Baroni et al. (1999) Liver 19:212-219; Lortat-Jacob et al. (1997) J. Hepatol. 26:894-903; Llorent et al. (1996) J. Hepatol. 24:555-563.

### SUMMARY OF THE INVENTION

The present invention provides methods of treating fibrotic diseases with a combination therapy of IFN-γ and pirfenidone or specific pirfenidone analogs. The methods generally involve administering to an individual suffering from a fibrotic disorder a therapeutically effective amount of IFN-γ in combination with a therapeutically effective amount of pirfenidone or a specific pirfenidone analog. In particular, the methods of the invention involve administering to an individual suffering from a fibrotic disorder a synergistic combination of IFN-i and pirfenidone or a specific pirfenidone analog.

### DEFINITIONS

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) increasing-survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “individual,” “host,” “subject,” and “patient,” used interchangeably herein, refer to a mammal, particularly a human.

The term “therapeutically effective amount” is meant an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art.

A “fibrotic condition,” “fibrotic disease” and “fibrotic disorder” are used interchangeably to refer to a condition, disease or disorder that is amenable to treatment by administration of a compound having anti-fibrotic activity. Fibrotic disorders include, but are not limited to, pulmonary fibrosis, including idiopathic pulmonary fibrosis (IPF) and pulmonary fibrosis from a known etiology, liver fibrosis, and renal-fibrosis. Other exemplary fibrotic conditions include musculoskeletal fibrosis, cardiac fibrosis, post-surgical adhesions, scleroderma, glaucoma, and skin lesions such as keloids.

A “specific pirfenidone analog,” and all grammatical variants thereof, refers to, and is limited to, each and every pirfenidone analog shown in Table 1.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise.

Thus, for example, reference to “a method” includes a plurality of such methods and reference to “an IFN-γ dose” includes reference to one or more doses and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating fibrotic diseases, including pulmonary fibrosis, idiopathic pulmonary fibrosis (IPF), pulmonary fibrosis from a known etiology, liver fibrosis, cardiac fibrosis, and renal fibrosis. The methods generally involve administering a therapeutically effective combination of IFN-γ and pirfenidone or a specific pirfenidone analog to an individual with a fibrotic disease. In particular, the methods of the invention involve administering to an individual suffering from fibrotic disease a synergistic combination of IFN-γ and pirfenidone or a specific pirfenidone analog.

Methods of Treating Fibrotic Diseases

The present invention provides methods for treating a fibrotic disorder in an individual having a fibrotic disorder. The method generally involves administering an effective amount of interferon-gamma (IFN-γ), and an effective amount of pirfenidone or a specific analog thereof. The methods provide for treatment of fibrotic diseases, including those affecting the lung such as idiopathic pulmonary fibrosis, pulmonary fibrosis from a known etiology, liver fibrosis or cirrhosis, cardiac and renal fibrosis. The etiology may be due to any acute or chronic insult including toxic, metabolic, genetic and infectious agents.

Fibrosis is generally characterized by the pathologic or excessive accumulation of collagenous connective tissue. Fibrotic disorders include, but are not limited to, collagen disease, interstitial lung disease, human fibrotic lung disease (e.g., obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, sarcoidosis, and the like), fibrotic vascular disease, arterial sclerosis, atherosclerosis, varicose veins, coronary infarcts, cerebral infarcts, myocardial fibrosis, musculoskeletal fibrosis, post-surgical adhesions, human kidney disease (e.g., nephritic syndrome, Alport's syndrome, HIV-associated nephropathy, polycystic kidney disease, Fabry's disease, diabetic nephropathy, chronic glomerulonephritis, nephritis associated with systemic lupus, and the like), cutis keloid formation, progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's opthalmopathy, diabetic retinopathy, glaucoma, Peyronie's disease, penis fibrosis, urethrostenosis after the test using a cystoscope, inner accretion after surgery, scarring, myelofibrosis, idiopathic retroperitoneal fibrosis, peritoneal fibrosis from a known etiology, drug-induced ergotism, fibrosis incident to benign or malignant cancer, fibrosis incident to microbial infection (e.g., viral, bacterial, parasitic, fungal, etc.), Alzheimer's disease, fibrosis incident to inflammatory bowel disease (including stricture formation in Crohn's disease and microscopic colitis), fibrosis induced by chemical or environmental insult (e.g., cancer chemotherapy, pesticides, radiation (e.g., cancer radiotherapy), and the like), and the like.

In some embodiments, an effective amount of IFN-γ and an effective amount of pirfenidone or a specific pirfenidone analog are amounts that, when administered in combination therapy, are effective to reduce fibrosis by at least about 10%, at least about 15%, at least about 20%, at least about 25%, at least about 30%, at least about 35%, at least about 40%, at least about 45%, or at least about 50%, or more, compared with the degree of fibrosis in the individual prior to treatment with the combination therapy.

In some embodiments, an effective amount of IFN-γ and an effective amount of pirfenidone or a specific pirfenidone analog are amounts that, when administered in combination therapy, are effective to increase at least one function of the organ affected by fibrosis (e.g., lung, liver, kidney, etc.) by at least about 10%, at least about 15%, at least about 20%, at least about 25%, at least about 30%, at least about 35%, at least about 40%, at least about 45%, or at least about 50%, or more, compared to the basal level of organ function in the individual prior to treatment with the combination therapy.

Methods of measuring the extent of fibrosis in a given organ, and methods of measuring the function of any given organ, are well known in the art.

In some embodiments, the present invention provides methods of treating a fibrotic condition that involve administering a synergistic combination of IFN-γ and pirfenidone or specific pirfenidone analog. As used herein, a “synergistic combination” of IFN-γ and pirfenidone or a specific pirfenidone analog is a combined dosage that is more effective in the therapeutic or prophylactic treatment of a fibrotic condition than the incremental improvement in treatment outcome that could be predicted or expected from a merely additive combination of (i) the therapeutic or prophylactic benefit of IFN-γ when administered at that same dosage as a monotherapy and (ii) the therapeutic or prophylactic benefit of pirfenidone or a specific pirfenidone analog when administered at the same dosage as a monotherapy.

The invention also provides a method for treatment of a fibrotic disease, such as IPF, liver fibrosis or renal fibrosis, in an individual comprising administering to the individual a combination of IFN-γ and pirfenidone or a specific pirfenidone analog that is effective for prophylaxis or therapy of fibrotic disease in the individual, e.g., increasing the probability of survival, reducing the risk of death, ameliorating the disease burden or slowing the progression of disease in the individual, while reducing the incidence or severity of one or more side effects that would ordinarily arise from treatment with an effective amount of IFN-γ or pirfenidone or a specific pirfenidone analog alone.

Methods of Treating Idiopathic Pulmonary Fibrosis

The present invention provides methods of treating idiopathic pulmonary fibrosis (IPF). The methods generally involve administering to an individual having IPF a combination of an effective amount of IFN-γ and an effective amount of pirfenidone or a specific pirfenidone analog.

In some embodiments, a diagnosis of IPF is confirmed by the finding of usual interstitial pneumonia (UIP) on histopathological evaluation of lung tissue obtained by surgical biopsy. The criteria for a diagnosis of IPF are known. Ryu et al. (1998) Mayo Clin.

Proc. 73:1085-1101.

In other embodiments, a diagnosis of IPF is a definite or probable IPF made by high resolution computer tomography (HRCT). In a diagnosis by HRCT, the presence of the following characteristics is noted: (1) presence of reticular abnormality and/or traction bronchiectasis with basal and peripheral predominance; (2) presence of honeycombing with basal and peripheral predominance; and (3) absence of atypical features such as micronodules, peribronchovascular nodules, consolidation, isolated (non-honeycomb) cysts, ground glass attenuation (or, if present, is less extensive than reticular opacity), and mediastinal adenopathy (or, if present, is not extensive enough to be visible on chest x-ray). A diagnosis of definite IPF is made if characteristics (1), (2), and (3) are met. A diagnosis of probable IPF is made if characteristics (1) and (3) are met.

In some embodiments, an “effective amount” of IFN-γ in combination with an “effective amount” of pirfenidone or a specific pirfenidone analog is a dosage combination that is effective to decrease disease progression by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or more, compared with a placebo control or an untreated control.

In other embodiments, the present invention provides methods that involve administering a synergistic combination of IFN-γ and pirfenidone or specific pirfenidone analog. As used herein, a “synergistic combination” of IFN-γ and pirfenidone or specific pirfenidone analog is a combined dosage that is more effective in the therapeutic or prophylactic treatment of IPF than the incremental improvement in treatment outcome that could be predicted or expected from a merely additive combination of (i) the therapeutic or prophylactic benefit of IFN-γ when administered at that same dosage as a monotherapy and (ii) the therapeutic or prophylactic benefit of pirfenidone or a specific pirfenidone analog when administered at the same dosage as a monotherapy.

The invention also provides a method for treatment of IPF in an individual comprising administering to the individual a combination of IFN-γ and pirfenidone or a specific pirfenidone analog that is effective for prophylaxis or therapy of IPF in the individual, e.g., increasing the probability of survival, reducing the risk of death, ameliorating the disease burden or slowing the progression of disease in the individual, while reducing the incidence or severity of one or more side effects that would ordinarily arise from treatment with an effective amount of IFN-γ or pirfenidone or a specific pirfenidone analog alone.

Disease progression is the occurrence of one or more of the following: (1) a decrease in predicted FVC of 10% or more; (2) an increase in A-a gradient of 5 mm Hg or more; (3) a decrease of 15% of more in single breath DL_{co}. Whether disease progression has occurred is determined by measuring one or more of these parameters on two consecutive occasions 4 to 14 weeks apart, and comparing the value to baseline.

Thus, e.g., where an untreated or placebo-treated individual exhibits a 50% decrease in FVC over a period of time, an individual administered with an effective combination of IFN-γ and pirfenidone or specific pirfenidone analog exhibits a decrease in FVC of 45%, about 42%, about 40%, about 37%, about 35%, about 32%, about 30%, or less, over the same time period.

In some embodiments, an “effective amount” of IFN-γ in combination with an effective amount of pirfenidone or a specific pirfenidone analog is a dosage combination that is effective to increase progression-free survival time, e.g., the time from baseline (e.g., a time point from 1 day to 28 days before beginnig of treatment) to death or disease progression is increased by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared a placebo-treated or an untreated control individual. Thus, e.g., in some embodiments an effective amount of IFN-γ in combination with an effective amount of pirfenidone or a specific pirfenidone analog is a dosage combination that is effective to increase the progression-free survival time by at least about 1 week, at least about 2 weeks, at least about 3 weeks, at least about 4 weeks, at least about 2 months, at least about 3 months, at least about 4 months, at least about 5 months, at least about 6 months, at least about 8 months, at least about 10 months, at least about 12 months, at least about 18 months, at least about 2 years, at least about 3 years, or longer, compared to a placebo-treated or untreated control.

In some embodiments, an effective amount of IFN-γ in combination with an effective amount of pirfenidone or a specific pirfenidone analog is a dosage combination that is effective to increase at least one parameter of lung function, e.g., an effective amount of a combination of IFN-γ and pirfenidone or a specific pirfenidone analog increases at least one parameter of lung function by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to an untreated individual or a placebo-treated control individual. In some of these embodiments, a determination of whether a parameter of lung function is increased is made by comparing the baseline value with the value at any time point after the beginning of treatment, e.g., 48 weeks after the beginning of treatment, or between two time points, e.g., about 4 to about 14 weeks apart, after the beginning of treatment.

In some embodiments, an effective amount of IFN-γ in combination with an effective amount of pirfenidone or a specific pirfenidone analog is a dosage combination that is effective to increase the FVC by at least about 10% at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more compared to baseline on two consecutive occasions 4 to 14 weeks apart.

In some of these embodiments, an effective amount of IFN-γ in combination with an effective amount of pirfenidone or a specific pirfenidone analog is a dosage combination that results in a decrease in alveolar:arterial (A-a) gradient of at least about 5 mm Hg, at least about 7 mm Hg, at least about 10 mm Hg, at least about 12 mm Hg, at least about 15 mm Hg, or more, compared to baseline.

In some of these embodiments, an effective amount of IFN-γ in combination with an effective amount of pirfenidone or a specific pirfenidone analog is a dosage combination that increases the single breath DL_{CO} by at least about 15%, at least about 20%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to baseline. CL_{Co} is the lung diffusing capacity for carbon monoxide, and is expressed as mL CO/mm Hg/second.

Parameters of lung function include, but are not limited to, forced vital capacity (FVC); forced expiratory volume (FEV₁); total lung capacity; partial pressure of arterial oxygen at rest; partial pressure of arterial oxygen at maximal exertion.

Lung function can be measured using any known method, including, but not limited to spirometry.

Methods of Treating Liver Fibrosis

The present invention provides methods of treating liver fibrosis, including reducing clinical liver fibrosis, reducing the likelihood that liver fibrosis will occur, and reducing a parameter associated with liver fibrosis. The methods generally involve administering a combination of an effective amount of IFN-γ and an effective amount of pirfenidone or specific pirfenidone analog to an individual in need thereof. In particular, the invention provides methods for treatment of liver fibrosis comprising administering a synergistic combination of IFN-γ and pirfenidone or specific pirfenidone analog to a patient in need thereof. Of particular interest in many embodiments is treatment of humans.

Liver fibrosis is a precursor to the complications associated with liver cirrhosis, such as portal hypertension, progressive liver insufficiency, and hepatocellular carcinoma. A reduction in liver fibrosis thus reduces the incidence of such complications. Accordingly, the present invention further provides methods of reducing the likelihood that an individual will develop complications associated with cirrhosis of the liver.

The present methods generally involve administering a therapeutically effective combination of IFN-γ and pirfenidone or specific pirfenidone analog. As used herein, an “effective amount” of IFN-γ in combination with an “effective amount” of pirfenidone or specific pirfenidone analog is a combined dosage of IFN-γ and pirfenidone or specific pirfenidone analog that is effective in reducing liver fibrosis; and/or that is effective in reducing the likelihood that an individual will develop liver fibrosis; and/or that is effective in reducing a parameter associated with liver fibrosis; and/or that is effective in reducing a disorder associated with cirrhosis of the liver.

In other embodiments, the present invention provides methods that involve administering a synergistic combination of IFN-γ and pirfenidone or specific pirfenidone analog. As used herein, a “synergistic combination” of IFN-γ and pirfenidone or specific pirfenidone analog is a combined dosage that is more effective in the therapeutic or prophylactic treatment of liver fibrosis than the incremental improvement in treatment outcome that could be predicted or expected from a merely additive combination of (i) the therapeutic or prophylactic benefit of IFN-γ when administered at that same dosage as a monotherapy and (ii) the therapeutic or prophylactic benefit of pirfenidone or a specific pirfenidone analog when administered at the same dosage as a monotherapy.

The invention also provides a method for treatment of liver fibrosis in an individual comprising administering to the individual a combination of IFN-γ and pirfenidone or a specific pirfenidone analog that is effective for prophylaxis or therapy of liver fibrosis in the individual, e.g., increasing the probability of survival, reducing the risk of death, ameliorating the disease burden or slowing the progression of disease in the individual, while reducing the incidence or severity of one or more side effects that would ordinarily arise from treatment with an effective amount of IFN-γ or pirfenidone or a specific pirfenidone analog alone.

Whether treatment with a combination of IFN-γ and pirfenidone or specific pirfenidone analog is effective in reducing liver fibrosis is determined by any of a number of well-established techniques for measuring liver fibrosis and liver function. Whether liver fibrosis is reduced is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis;

score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P—P) bridging; stage 4, Fibrous expansion of portal areas with marked bridging (P—P) as well as portal-central (P—C); stage 5, Marked bridging (P—P and/or P—C) with occasional nodules (incomplete cirrhosis); stage 6, Cirrhosis, probable or definite. The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective combination of IFN-γ and pirfenidone or a specific pirfenidone analog is a combined dosage amount of IFN-γ and pirfenidone or a specific pirfenidone analog that effects a change of one unit or more in the fibrosis stage based on pre- and post-therapy liver biopsies. In particular embodiments, a therapeutically effective combination of IFN-γ and pirfenidone or a specific pirfenidone analog reduces liver fibrosis by at least one unit in the METAVIR, the Knodell, the Scheuer, the Ludwig, or the Ishak scoring system.

Secondary, or indirect, indices of liver function can also be used to evaluate the efficacy of IFN-γ and pirfenidone or specific pirfenidone analog combination treatment. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

In another embodiment, an effective combination of IFN-γ and pirfenidone or a specific pirfenidone analog is an amount of IFN-γ and an amount of pirfenidone or a specific pirfenidone analog that in combination are effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or in a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procoilagen I peptide, and laminin. Additional biochemical markers of liver fibrosis include α-2-macroglobulin, haptoglobin, gamma globulin, apolipoprotein A, and gamma glutamyl transpeptidase.

In another embodiment, a therapeutically effective combination of IFN-γ and pirfenidone or a specific pirfenidone analog is an amount of IFN-γ and an amount of pirfenidone or a specific pirfenidone analog that in combination are effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or in a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., eruyme-linked immunosorbent assays (ELISA), radioimmunoassays, and the like, using antibody specific for a given serum marker.

Quantitative tests of functional liver reserve can also be used to assess the efficacy of treatment with IFN-γ. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

In another embodiment, a therapeutically effective combination of IFN-γ and pirfenidone or a specific pirfenidone analog is an amount of IFN-γ and an amount of pirfenidone or specific pirfenidone analog that in combination are effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or in a placebo-treated individual.

Whether combination therapy with IFN-γ and pirfenidone or a specific pirfenidone analog is effective in reducing the incidence of a disorder associated with cirrhosis of the liver can readily be determined by those skilled in the art.

Reduction in liver fibrosis increases liver function. Thus, the invention provides methods for increasing liver function, generally involving administering a therapeutically effective combination of IFN-γ and pirfenidone or a specific pirfenidone analog. Liver fumctions include, but are not limited to, synthesis of proteins such as serum proteins (e.g., albumin, clotting factors, alkaline phosphatase, aminotrnsferases (e.g., alanine transaminase, aspartate transaminase), 5′-nucleosidase, γ-glutauinyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

In another embodiment, a therapeutically effective combination of IFN-γ with pirfenidone or a specific pirfenidone analog is one that is effective to increase liver function by at least about 10%, at least about 20%, at least about 30%, at least about 40%, at least about 50%, at least about 60%, at least about 70%, at least about 80%, or more. For example, a therapeutically effective combination of IFNγ and pirfenidone or a specific pirfenidone analog is a combined dosage effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective combination of IFNγ with pirfenidone or a specific pirfenidone analog is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver flnction to within a normal range.

Methods of Treating Renal Fibrosis

Renal fibrosis is characterized by the excessive accumulation of extracellular matrix (ECM) components. Overproduction of transforming growth factor-beta (TGF-β) is believed to underly tissue fibrosis caused by excess deposition of ECM, resulting in disease. TGF-β's fibrogenic action results from simultaneous stimulation of matrix protein synthesis, inhibition of matrix degradation and enhanced integrin expression that facilitates ECM assembly.

The present invention provides methods of treating renal fibrosis. The methods generally involve administering to an individual having renal fibrosis a combination of an effective amount of IFN-γ and an effective amount of pirfenidone or a specific pirfenidone analog. As used herein, an “effective amount” of IFN-γ in combination with an “effective amount” of pirfenidone or specific pirfenidone analog is a combined dosage of IFN-γ and pirfenidone or specific pirfenidone analog that is effective in reducing renal fibrosis; and/or that is effective in reducing the likelihood that an individual will develop renal fibrosis; and/or that is effective in reducing a parameter associated with renal fibrosis; and/or that is effective in reducing a disorder associated with fibrosis of the kidney.

In one embodiment, an effective combination of IFN-γ and pirfenidone or a specific pirfenidone analog is an amount of IFN-γ and an amount of pirfenidone or a pirfenidone analog that in combination are sufficient to reduce renal fibrosis by at least about 10%, at least about 15%, at least about 20%, at least about 25%, at least about 30%, at least about 35%, at least about 40%, at least about 45%, at least about 50%, compared to the degree of renal fibrosis in the individual prior to treatment with the combination therapy of the present invention.

Whether fibrosis is reduced in the kidney is determined using any known method. For example, histochemical analysis of kidney biopsy samples for the extent of ECM deposition and/or fibrosis is performed. Other methods are known in the art. See, e.g., Masseroli et al. (1998) Lab. Invest. 78:511-522; U.S. Pat. No. 6,214,542.

In some embodiments, an effective combination of IFN-γ and pirfenidone or a specific pirfenidone analog is an amount of IFN-γ and an amount of pirfenidone or a specific pirfenidone analog that in combination are effective to increase kidney function by at least about 10%, at least about 15%, at least about 20%, at least about 25%, at least about 30%, at least about 35%, at least about 40%, at least about 45%, at least about 50%, compared to the basal level of kidney function in the individual prior to treatment with the combination therapy of the present invention.

In some embodiments, an effective combination of IFN-γ and pirfenidone or a specific pirfenidone analog is an amount of IFN-γ and an amount of pirfenidone or a specific pirfenidone analog that in combination are effective to slow the decline in kidney function by at least about 10%, at least about 15%, at least about 20%, at least about 25%, at least about 30%, at least about 35%, at least about 40%, at least about 45%, at least about 50%, compared to the decline in kidney function that would occur in the absence of treatment with the combination therapy of the present invention.

Kidney function can be measured using any known assay, including, but not limited to, plasma creatinine level (where normal levels are generally in a range of from about 0.6 to about 1.2 mg/dL); creatinine clearance (where the normal range for creatinine clearance is from about 97 to about 137 mL/minute in men, and from about 88 to about 128 mL/minute in women); the glomerular filtration rate (either calculated or obtained from inulin clearance or other methods), blood urea nitrogen (where the normal range is from about 7 to about 20 mg/dL); and urine protein levels.

In other embodiments, the present invention provides methods that involve administering a synergistic combination of IFN-γ and pirfenidone or a specific pirfenidone analog. As used herein, a “synergistic combination” of IFN-γ and pirfenidone or a specific pirfenidone analog is a combined dosage that is more effective in the therapeutic or prophylactic treatment of renal fibrosis than the incremental improvement in treatment outcome that could be predicted or expected from a merely additive combination of (i) the therapeutic or prophylactic benefit of IFN-γ when administered at that same dosage as a monotherapy and (ii) the therapeutic or prophylactic benefit of pirfenidone or a specific pirfenidone analog when administered at the same dosage as a monotherapy.

The invention also provides a method for treatment of renal fibrosis in an individual comprising administering to the individual a combination of IFN-γ and pirfenidone or a specific pirfenidone analog that is effective for prophylaxis or therapy of renal fibrosis in the individual, e.g., increasing the time to doubling of serum creatinine levels, increasing the time to end-stage renal disease requiring renal replacement therapy (e.g., dialysis or transplant), increasing the probability of survival, reducing the risk of death, ameliorating the disease burden or slowing the progression of disease in the individual, while reducing the incidence or severity of one or more side effects that would ordinarily arise from treatment with an effective amount of IFN-γ or pirfenidone or a specific pirfenidone analog alone.

Without being limited by the following description, the mode of operation of the active ingredients of the combination therapy of the invention is postulated to be the following. The IFN-γ may modify immune action, regulating the synthesis of other cytokines such as TGFβ and inhibiting fibroblast proliferation and or migration. Pirfenidone, on the other hand, may effectively inhibit the synthesis and deposition of ECM by activated fibroblasts. In either case, the end point will be the same, namely the therapeutic or prophylactic treatment of fibrotic disease.

Pirfenidone and Analogs Thereof

Pirfenidone(5-methyl-1-phenyl-2-(1H)-pyridone) and specific pirfenidone analogs are disclosed for the treatment of fibrotic conditions. A “fibrotic condition” is one that is amenable to treatment by administration of a compound having anti-fibrotic activity. 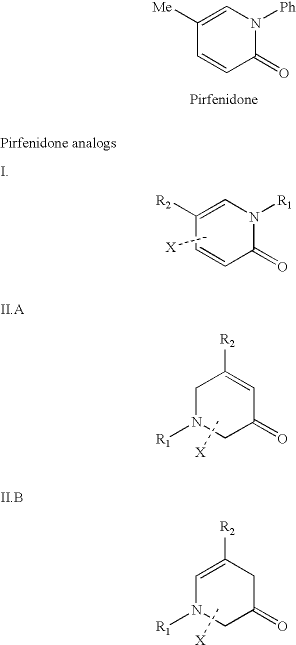
Descriptions for Substituents R₁, R₂, X

R₁: carbocyclic (saturated and unsaturated), heterocyclic (saturated or unsaturated), alkyls (saturated and unsaturated). Examples include phenyl, benzyl, pyrimidyl, naphthyl, indolyl, pyrrolyl, furyl, thienyl, imidazolyl, cyclohexyl, piperidyl, pyrrolidyl, morpholinyl, cyclohexenyl, butadienyl, and the like.

R₁ can further include substitutions on the carbocyclic or heterocyclic moieties with substituents such as halogen, nitro, amino, hydroxyl, alkoxy, carboxyl, cyano, thio, alkyl, aryl, heteroalkyl, heteroaryl and combinations thereof, for example, 4-nitrophenyl, 3-chlorophenyl, 2,5-dinitrophenyl, 4-methoxyphenyl, 5-methyl-pyrrolyl, 2,5-dichlorocyclohexyl, guanidinyl-cyclohexenyl and the like.

R₂: alkyl, carbocylic, aryl, heterocyclic. Examples include: methyl, ethyl, propyl, isopropyl, phenyl, 4-nitrophenyl, thienyl and the like.

X: may be any number (from 1 to 3) of substituents on the carbocyclic or heterocyclic ring. The substituents can be the same or different. Substituents can include hydrogen, alkyl, heteroalkyl, aryl, heteroaryl, halo, nitro, carboxyl, hydroxyl, cyano, amino, thio, alkylamino, haloaryl and the like.

The substituents may be optionally further substituted with 1-3 substituents from the group consisting of alkyl, aryl, nitro, alkoxy, hydroxyl and halo groups. Examples include: methyl, 2,3-dimethyl, phenyl, p-tolyl, 4-chlorophenyl, 4-nitrophenyl, 2,5-dichlorophenyl, furyl, thienyl and the like.

Specific Examples include:

TABLE 1

| IA | IIB |
|---|---|
| 5-Methyl-1-(2′-pyridyl)-2-(1H) pyridine, | 6-Methyl-1-phenyl-3-(1H) pyridone, |
| 6-Methyl-1-phenyl-2-(1H) pyridone, | 5-Methyl-1-p-tolyl-3-(1H) pyridone, |
| 5-Methyl-3-phenyl-1-(2′-thienyl)-2-(1H) | 5-Methyl-1-(2′-naphthyl)-3-(1H) pyridone, |
| pyridone, | |
| 5-Methyl-1-(2′-naphthyl)-2-(1H) pyridone, | 5-Methyl-1-phenyl-3-(1H) pyridone, |
| 5-Methyl-1-p-tolyl-2-(1H) pyridone, | 5-Methyl-1-(5′-quinolyl)-3-(1H) pyridone, |
| 5-Methyl-1-(1′naphthyl)-2-(1H) pyridone, | 5-Ethyl-1-phenyl-3-(1H) pyridone, |
| 5-Ethyl-1-phenyl-2-(1H) pyridone, | 5-Methyl-1-(4′-methoxyphenyl)-3-(1H) pyridone, |
| 5-Methyl-1-(5′-quinolyl)-2-(1H) pyridone, | 4-Methyl-1-phenyl-3-(1H) pyridone, |
| 5-Methyl-1-(4′-quinolyl)-2-(1H) pyridone, | 5-Methyl-1-(3′-pyridyl)-3-(1H) pyridone, |
| 5-Methyl-1-(4′-pyridyl)-2-(1H) pyridone, | 5-Methyl-1-(2′-Thienyl)-3-(1H) pyridone, |
| 3-Methyl-1-phenyl-2-(1H) pyridone, | 5-Methyl-1-(2′-pyridyl)-3-(1H) pyridone, |
| 5-Methyl-1-(4′-methoxyphenyl)-2-(1H) | 5-Methyl-1-(2′-quinolyl)-3-(1H) pyridone, |
| pyridone, | |
| 1-Phenyl-2-(1H) pyridone, | 1-Phenyl-3-(1H) pyridine, |
| 1,3-Diphenyl-2-(1H) pyridone, | 1-(2′-Furyl)-5-methyl-3-(1H) pyridone, |
| 1,3-Diphenyl-5-methyl-2-(1H) pyridone, | 1-(4′-Chlorophenyl)-5-methyl-3-(1H) pyridine. |
| 5-Methyl-1-(3′-trifluoromethylphenyl)-2- | |
| (1H)-pyridone, | |
| 3-Ethyl-1-phenyl-2-(1H) pyridone, | |
| 5-Methyl-1-(3′-pyridyl)-2-(1H) pyridone, | |
| 5-Methyl-1-(3-nitrophenyl)-2-(1H) | |
| pyridone, | |
| 3-(4′-Chlorophenyl)-5-Methyl-1-phenyl-2- | |
| (1H) pyridone, | |
| 5-Methyl-1-(2′-Thienyl)-2-(1H) pyridone, | |
| 5-Methyl-1-(2′-thiazolyl)-2-(1H) pyridone, | |
| 3,6-Dimethyl-1-phenyl-2-(1H) pyridone, | |
| 1-(4′Chlorophenyl)-5-Methyl-2-(1H) | |
| pyridone, | |
| 1-(2′-Imidazolyl)-5-Methyl-2-(1H) | |
| pyridone, | |
| 1-(4′-Nitrophenyl)-2-(1H) pyridone, | |
| 1-(2′-Furyl)-5-Methyl-2-(1H) pyridone, | |
| 1-Phenyl-3-(4′-chlorophenyl)-2-(1H) | |
| pyridine. | |

U.S. Pat. Nos. 3,974,281; 3,839,346; 4,042,699; 4,052,509; 5,310,562; 5,518,729; 5,716,632; and 6,090,822 describe methods for the synthesis and formulation of pirfenidone and specific pirfenidone analogs in pharmaceutical compositions suitable for use in the methods of the present invention.

Interferon-Gamma

The nucleic acid sequences encoding IFN-γ polypeptides may be accessed from public databases, e.g. Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J. Biol. Chem. 259:6790.

IFN-γ1b (Actimmune®; human interferon) is a single-chain polypeptide of 140 amino acids. It is made recombinantly in E. coli and is unglycosylated. Rinderknecht et al. (1984) J. Biol. Chem. 259:6790-6797.

The IFN-γ to be used in the compositions of the present invention may be any of natural IFN-γs, recombinant IFN-γs and the derivatives thereof so far as they have a IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra. consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG which, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925, herein incorporated by reference. Native IFN-γ moieties include molecules variously extending from amino acid residues 24-150; 24-151, 24-152; 24- 153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. In one embodiment, the invention contemplates the use of IFN-γ variants with one or more non-naturally occurring glycosylation and/or pegylation sites that are engineered to provide glycosyl- and/or PEG-derivatized polypeptides with reduced serum clearance, such as the IFN-γ polypeptide variants described in International Patent Publication No. WO 01/36001. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Dosages, Formulations, and Routes of Administration

IFN-γ and pirfenidone or specific pirfenidone analogs are administered to individuals in a formulation (e.g., in separate formulations) with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In the subject methods, the active agent(s) may be administered to the host using any convenient means capable of resulting in the desired therapeutic effect. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Effective dosages of IFN-γ can range from about 0.5 μg/m² to about 500 μg/m², usually from about 1.5 μg/m² to 200 μg/m², depending on the size of the patient. This activity is based on 10⁶ international units (IU) per 50 μg of protein.

Those of skill will readily appreciate that dose levels can vary as a function of the specific compound, the severity of the symptoms and the susceptibility of the subject to side effects. Preferred dosages for a given compound are readily determinable by those of skill in the art by a variety of means. A preferred means is to measure the physiological potency of a given compound.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m²,

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide (e.g., a polynucleotide encoding IFN-γ), it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells. Of particular interest in these embodiments is use of a liver-specific promoter to drive transcription of an operably linked IFN-γ coding sequence preferentially in liver cells.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

In particular embodiments of interest, IFN-γ is administered as a solution suitable for subcutaneous injection. For example, IFN-γ is in a formulation containing 40 mg mannitol/mL, 0.72 mg sodium succinate/mL, 0.10 mg polysorbate 20/mL. In particular embodiments of interest, IFN-γ is administered in single-dose forms of 200 μg/dose subcutaneously.

Multiple doses of IFN-γ can be administered, e.g., IFN-γ can be administered once per month, twice per month, three times per month, once per week, twice per week, three times per week, four times per week, five times per week, six times per week, or daily, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In particular embodiments of interest, IFN-γ is administered three times per week over a period of about 48 weeks.

Pirfenidone can be administered once per month, twice per month, three times per month, once per week, twice per week, three times per week, four times per week, five times per week, six times per week, daily, or in divided daily doses ranging from once daily to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Effective dosages of pirfenidone or specific pirfenidone analogs will range from about 5 mg/kg/day to about 125 mg/kg/day, or at a fixed dosage of about 400 mg to about 3600 mg per day, administered orally. Other doses and formulations of pirfenidone and specific pirfenidone analogs suitable for use in the treatment of fibrotic diseases are described in U.S. Pat. Nos. 3,974,281; 3,839,346; 4,042,699; 4,052,509; 5,310,562; 5,518,729; 5,716,632; and 6,090,822.

IFN-γ and pirfenidone (or pirfenidone analog) are generally administered in separate formulations. IFN-γ and pirfenidone (or pirfenidone analog) may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

In one embodiment, the invention provides a method for treatment of a fibrotic disease in an individual comprising administering to the individual a combination therapy comprising about 25 mcg/m² (25 μg/m²) to about 50 mcg/m² IFN-γ three times weekly and about 2400 mg to about 3600 mg pirfenidone or a specific pirfenidone analog daily. In another embodiment, the invention provides a method for treatment of a fibrotic disease in an individual comprising administering to the individual a combined dosage of about 50 μg to about 100 μg IFN-γ three times weekly and about 400 mg to about 2400 mg pirfenidone or a specific pirfenidone analog daily.

In one embodiment, the invention provides a method for treatment of IPF in an individual comprising administering to the individual a combination therapy comprising about 25 mcg/m² to about 50 mcg/m² IFN-γ three times weekly and about 2400 mg to about 3600 mg pirfenidone or a specific pirfenidone analog daily. In another embodiment, the invention provides a method for treatment of IPF in an individual comprising administering to the individual a combined dosage of about 50 μg to about 100 μg IFN-γ three times weekly and about 400 mg to about 2400 mg pirfenidone or a specific pirfenidone analog daily.

In one embodiment, the invention provides a method for treatment of liver fibrosis in an individual comprising administering to the individual a combination therapy comprising about 25 mcg/m² to about 50 mcg/m² IFN-γ three times weekly and about 2400 mg to about 3600 mg pirfenidone or a specific pirfenidone analog daily. In another embodiment, the invention provides a method for treatment of liver fibrosis in an individual comprising administering to the individual a combined dosage of about 50 μg to about 100 μg IFN-γ three times weekly and about 400 mg to about 2400 mg pirfenidone or a specific pirfenidone analog daily.

In one embodiment, the invention provides a method for treatment of renal fibrosis in an individual comprising administering to the individual a combination therapy comprising about 25 mcg/r² to about 50 mcg/m² IFN-γ three times weekly and about 2400 mg to about 3600 mg pirfenidone or a specific pirfenidone analog daily. In another embodiment, the invention provides a method for treatment of renal fibrosis in an individual comprising administering to the individual a combined dosage of about 50 μg to about 100 μg IFN-γ three times weekly and about 400 mg to about 2400 mg pirfenidone or a specific pirfenidone analog daily.

In an exemplary embodiment, the invention provides a method for treatment of a fibrotic disease in an individual, the method comprising administering to an individual having a fibrotic disease IFN-γ in an amount of 200 μg three times per week subcutaneously; and pirfenidone or a pirfenidone analog in an amount of 1800 mg/day to 3600 mg/day, in a single dose or in two or three divided doses administered orally per day, for a period of time of 2-5 years. In another exemplary embodiment, the invention provides a method for treatment of a fibrotic disease in an individual, the method comprising administering to an individual having a fibrotic disease IFN-γ in an amount of 200 μg three times per week subcutaneously; and pirfenidone or a pirfenidone analog in an amount of 1200 mg daily, in a single dose or in two or three divided doses administered orally per day, for a period of time of 2-5 years. For example, IFN-γ is administered as a solution suitable for subcutaneous injection. For example, IFN-γ is in a formulation containing 40 mg mannitol/mL, 0.72 mg sodium succinate/mL, 0.10 mg polysorbate 20/mL.

In another embodiment, the invention provides a method for treatment of fibrotic disease in an individual comprising administering to the individual a synergistic combination of (i) about IFN-γ 25 mcg/m² to about 100 mcg/m² IFN-γ, or about 50 μg IFN-γ to about 200 μg IFN-γ, administered subcutaneously three times per week and (ii) about 400 mg to about 3600 mg pirfenidone or a specific pirfenidone analog in a single dose or two or three divided doses administered orally per day.

In another exemplary embodiment, the invention provides a method for treatment of a IPF in an individual, the method comprising administering to an individual having IPF IFN-γ in an amount of 200 μg three times per week subcutaneously; and pirfenidone or a pirfenidone analog in an amount of 1800 mg/day to 3600 mg/day, in a single dose or in two or three divided doses administered orally per day, for a period of time of 2-5 years. In yet another exemplary embodiment, the invention provides a method for treatment of IPF in an individual, the method comprising administering to an individual having IPF IFN-γ in an amount of 200 μg three times per week subcutaneously; and pirfenidone or a pirfenidone analog in an amount of 1200 mg daily, in a single dose or in two or three divided doses administered orally per day, for a period of time of 2-5 years.

In another embodiment, the invention provides a method for treatment of IPF in an individual comprising administering to the individual a synergistic combination of (i) about IFN-γ 25 μg/m² to about 100 μg/m² IFN-γ, or about 50 μg IFN-γ to about 200 μg IFN-γ, administered subcutaneously three times per week and (ii) about 400 mg to about 3600 mg pirfenidone or a specific pirfenidone analog in a single dose or two or three divided doses administered orally per day.

In an exemplary embodiment, the invention provides a method for treatment of liver fibrosis in an individual, the method comprising administering to an individual having liver fibrosis IFN-γ in an amount of 200 μg three times per week subcutaneously; and pirfenidone or a pirfenidone analog in an amount of 1800 mg/day to 3600 mg/day, in a single dose or in two or three divided doses administered orally per day, for a period of time of 2-5 years. In another exemplary embodiment, the invention provides a method for treatment of liver fibrosis in an individual, the method comprising administering to an individual having liver fibrosis IFN-γ in an amount of 200 μg three times per week subcutaneously; and pirfenidone or a pirfenidone analog in an amount of 1200 mg daily, in a single dose or in two or three divided doses administered orally per day, for a period of time of 2-5 years.

In another embodiment, the invention provides a method for treatment of liver fibrosis in an individual comprising administering to the individual a synergistic combination of (i) about IFN-γ 25 mcg/m² to about 100 mcg/m² IFN-γ, or about 50 μg IFN-γ to about 200 μg IFN-γ, administered subcutaneously three times per week and (ii) about 400 mg to about 3600 mg pirfenidone or a specific pirfenidone analog in a single dose or two or three divided doses administered orally per day.

In an exemplary embodiment, the invention provides a method for treatment of renal fibrosis in an individual, the method comprising administering to an individual having renal fibrosis IFN-γ in an amount of 200 μg three times per week subcutaneously; and pirfenidone or a pirfenidone analog in an amount of 1200 mg/day to 3600 mg/day, in a single dose or in two or three divided doses administered orally per day, for a period of time of 2-5 years. In another exemplary embodiment, the invention provides a method for treatment of renal fibrosis in an individual, the method comprising administering to an individual having renal fibrosis IFN-γ in an amount of 200 μg three times per week subcutaneously; and pirfenidone or a pirfenidone analog in an amount of 800 mg daily, in a single dose or in two or three divided doses administered orally per day, for a period of time of 2-5 years.

In another embodiment, the invention provides a method for treatment of renal fibrosis in an individual comprising administering to the individual a synergistic combination of (i) about IFN-γ 25 mcg/m² to about 100 mcg/m² IFN-γ, or about 50 μg IFN-γ to about 200 μg IFN-γ, administered subcutaneously three times per week and (ii) about 400 mg to about 3600 mg pirfenidone or a specific pirfenidone analog in a single dose or two or three divided doses administered orally per day.

Additional Agents

In some embodiments, IFN-γ and pirfenidone or a specific pirfenidone analog are co-administered with one or more additional agents. Suitable additional agents include corticosteroids, such as prednisone. When co-administered with IFN-γ and pirfenidone or a specific pirfenidone analog in the treatment of a fibrotic disease, such as IPF, liver fibrosis, or renal fibrosis, prednisone can be administered in an amount of 7.5 mg or 15 mg daily, administered orally.

Subjects Suitable for Treatment

The subject methods are suitable for treatment of individuals diagnosed as having a fibrotic disease, such as IPF, liver fibrosis or renal fibrosis. The subject methods are also suitable for treatment of individuals who are at risk of developing a fibrotic disease.

Individuals with liver fibrosis who are suitable for treatment according to the methods of the invention include individuals who have been clinically diagnosed with liver fibrosis, as well as individuals who have not yet developed clinical liver fibrosis but who are considered at risk of developing liver fibrosis. Such individuals include, but are not limited to, individuals who are infected with HCV; individuals who are infected with HBV; individuals who are infected with Schistosoma mansoni; individuals who have been exposed to chemical agents known to result in liver fibrosis; individuals who have been diagnosed with Wilson's disease; individuals diagnosed with hemochromatosis; and individuals with alcoholic liver disease; individuals with non-alcoholic steatohepatitis; individuals with autoimmune hepatitis; individuals with primary sclerosing cholangitis, primary biliary cirrhosis, or alpha-1-antitrysin deficiency.

Individuals who have been clinically diagnosed as infected with HCV are of particular interest in many embodiments. Individuals who are infected with HCV are identified as having HCV RNA in their blood, and/or having anti-HCV antibody in their serum. In many embodiments, individuals of interest include those who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods of the present invention. In other embodiments, individuals suitable for treatment with the methods of the instant invention are patients with decompensated cirrhosis with clinical manifestations, including patients with far-advanced liver cirrhosis, including those awaiting liver transplantation. In still other embodiments, individuals suitable for treatment with the methods of the instant invention include patients with milder degrees of fibrosis including those with early fibrosis (stages 1 and 2 in the METAVIR, Ludwig, and Scheuer scoring systems; or stages 1, 2, or 3 in the Ishak scoring system.).

The subject methods are suitable for treatment of individuals diagnosed as having IPF. The methods are also suitable for treatment of individuals having IPF who were previously treated with corticosteroids within the previous 24 months, and who failed to respond to previous treatment with corticosteroids. Also included are subjects who have an FVC at the outset of treatment that is at least 55% of the predicted FVC. The percent predicted FVC values are based on normal values, which are known in the art. See, e.g., Crapo et al. (1981) Am. Rev. Respir. Dis. 123:659-664. FVC is measured using-standard methods of spirometry.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating a fibrotic disease in an individual, the method comprising administering to an individual suffering from a fibrotic disease a synergistic combination of IFN-γ and pirfenidone or a specific pirfenidone analog that is effective in the treatment or prophylaxis of the fibrotic disease in the individual.

2. A method of treating a fibrotic disease in an individual, the method comprising administering to an individual suffering from a fibrotic disease a combination of IFN-γ and pirfenidone or a specific pirfenidone analog that is effective in the treatment or prophylaxis of the fibrotic disease in the individual and that reduces the incidence or severity of one or more side effects ordinarily associated with the administration of either drug alone in the treatment of the fibrotic disease.

3. A method of treating a fibrotic disease in an individual, the method comprising administering to an individual suffering from a fibrotic disease a combined dosage of about 50 mcg to about 200 mcg IFN-γ three times weekly and about 400 mg to about 3600 mg pirfenidone or a specific pirfenidone analog daily.

4. The method of any of claims 1-3 wherein the fibrotic disease is pulmonary fibrosis.

5. The method of claim 4 wherein the pulmonary fibrosis is idiopathic pulmonary fibrosis.

6. The method of claim 4 wherein the pulmonary fibrosis is from a known etiology.

7. The method of any of claims 1-3 wherein the fibrotic disease is liver fibrosis.

8. The method of any of claims 1-3 wherein the fibrotic disease is renal fibrosis.

9. The method of any of claims 1-3 wherein the fibrotic disease is cardiac fibrosis.

10. The method of any of claims 1-3 wherein the fibrotic disease is scleroderma.

11. The method of any of claims 1-10 wherein the individual is a human.
